(19) United States
(12) Patent Application Publication
(10) Pub. No.: US 2010/0016947 A1
(43) Pub. Date: Jan. 21, 2010

(54) SYSTEMS AND METHODS FOR CREATING CUSTOMIZED ENDOVASCULAR STENTS AND STENT GRAFTS

(76) Inventor: John Daniel Dobak, III, La Jolla, CA (US)

Correspondence Address: MAYER & WILLIAMS PC, 251 NORTH AVENUE WEST, 2ND FLOOR, WESTFIELD, NJ, 07090, US

(21) Appl. No.: 12/566,622

(22) Filed: Sep. 24, 2009

Related U.S. Application Data
(63) Continuation of application No. 11/796,128, filed on Apr. 26, 2007, now abandoned.
(60) Provisional application No. 60/795,779, filed on Apr. 28, 2006.

(51) Int. Cl. A61F 2/06; B21F 1/00

(52) U.S. Cl. 623/1.13; 140/92.1

(57) ABSTRACT

A system and method are provided for making a customized stent or stent graft, including the steps of obtaining a digital image of the endoluminal shape of an artery or the blood flow channel of an aneurysm, processing the obtained image to create a three dimensional model of the shape or channel, and fabricating a scaffold around the model such that the scaffold substantially conforms to the model.

### STATEMENT OF RELATED APPLICATIONS

This application is a continuation of U.S. patent application Ser. No. 11/796,128, filed Apr. 26, 2007, entitled “Systems and Methods For Creating Customized Endovascular Stents and Stent Grafts,” which claims the benefit of U.S. Provisional Patent Application Ser. No. 60/795,779, filed Apr. 28, 2006, entitled “Methods for Creating Customized Endovascular Stents and Stent Grafts.” Both of the prior applications are incorporated herein by reference in their entireties.

### BACKGROUND

Endovascular grafts are tubular structures used to prop open and restore blood flow in arteries. In the case of abdominal aortic aneurysm (AAA) grafts they can prevent the rupture of the aneurysms. Stents and stent grafts may also be placed near or across the opening of intracranial aneurysms to redirect or reduce blood flow and flow streams into the saccular aneurysm. The stents may also be used to keep occlusion coils from extending into the parent vessel.

One problem with current endovascular grafts and stents is a lack of conformation with the lumen of the vessel into which they are placed. Vessels may be curved, or tortuous, bifurcated, and can have changing diameter. Current stents and grafts differ in their flexibility and ability to conform to the vessel anatomy. This can lead to several problems.

In the case of AAA endoluminal grafts, leakage of blood between the vessel wall and the graft is relatively common and can lead to death or require surgical repair. In addition, it the distance between the renal artery branches from the aorta to the aneurysm may vary and typical stent grafts may occlude these arteries if the stent is too long. Alternatively, the stent may not be secured well if the distance is very short. Customized stents may resolve this issue.

Coronary stents are often coated with drugs to inhibit re-stenosis of the vessel. Delivery of drug into the endovascular tissue is governed by the contact of the stent with the vessel wall and poor conformation/contact can lead to poor drug delivery. Overexpansion of the graft or stent during deployment is sometimes used to improve conformation and contact to the anatomy of the vessel, and this can damage the vessel and induce the processes that lead to restonosis. Lastly, vessels, such as those in the brain, are fragile and may tear or dissect when rigid non-conforming stents are placed and/or overexpanded. It would therefore be desirable to have a stent or endovascular graft that can be fabricated to conform to the specific anatomy of an individual patient.

Stents and stent grafts (also known as endografts) are fabricated to expand from a small diameter to a large diameter through a self-expansion design or through balloon deployment. Currently many stents are fabricated by laser from a hollow thin walled tube of nitinol. A lattice like pattern is cut into the tube which allows the tube to be expanded. Similar patterns may be photo etched into thin sheets of metal. Wire braids may be employed or wire bending techniques. Typically different diameter devices are made; however, these devices are not made to conform to an individual patient's vascular anatomy.

### SUMMARY

In one aspect, the invention is directed to a method for making a customized stent or stent graft, including the steps of: obtaining a digital image of the endoluminal shape of an artery or the blood flow channel of an aneurysm; processing the obtained image to create a three dimensional model of the shape or channel; and fabricating a scaffold around the model such that the scaffold substantially conforms to the model.

Implementations of the method may include one or more of the following. The digital image may be three-dimensional, and the three-dimensional model may be created by stereolithography. The scaffold may be a wire scaffold. The processing may include etching, and the scaffold may further be sterilized. The scaffold may be formed in a braided pattern or a V-shaped pattern. The scaffold may be a helix, where the helix is formed by a wire, such as a flat or round wire. The scaffold may be drug-coated, and a graft material such as nylon, Teflon®, or Gore-Tex® may form this graft material. A hole may be etched into the scaffold, such as to contain a drug. Struts or hooks may be mounted to the scaffold. The scaffold may be created in a modular fashion where the modules are connected together into a unitary component prior to or during installation in a patient.

In another aspect, the invention is directed towards a stent graft or stent created by the above process. In a further aspect, the invention is directed towards a computer-readable medium containing instructions for causing a computer to implement the above method.

### BRIEF DESCRIPTION OF THE DRAWINGS

FIG. 1 illustrates a flowchart of a method for fabricating custom stents or stent grafts, in particular for an AAA.

FIG. 2 illustrates a three-dimensional model with different collapsible scaffolding configurations.

FIG. 3 illustrates a pattern to create a continuous V-shaped scaffold.

### DETAILED DESCRIPTION

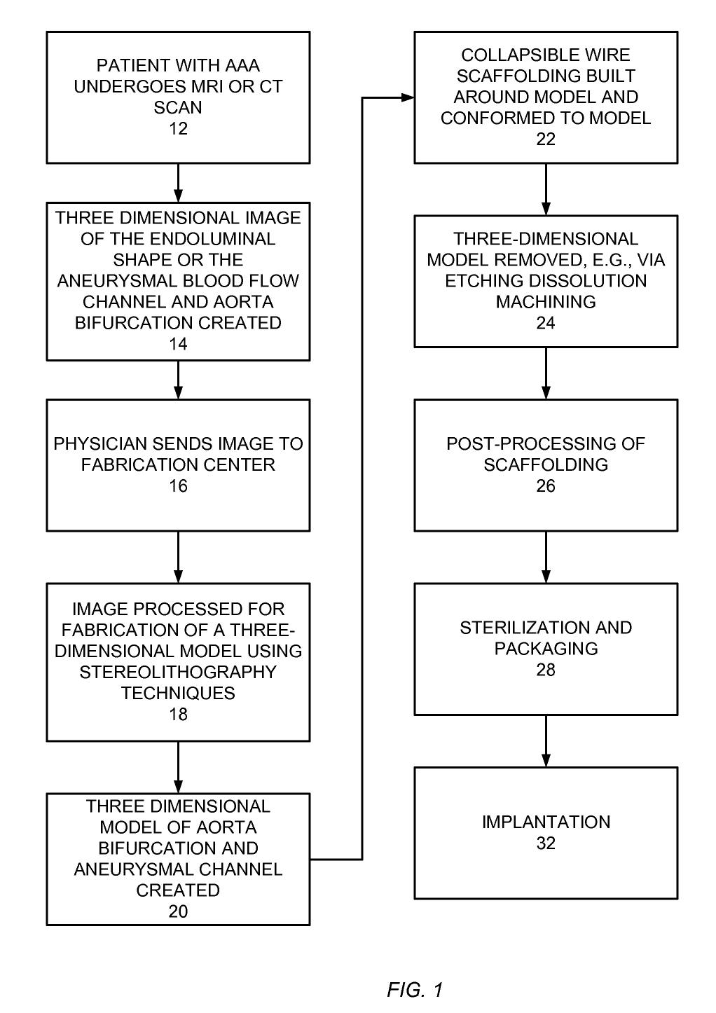

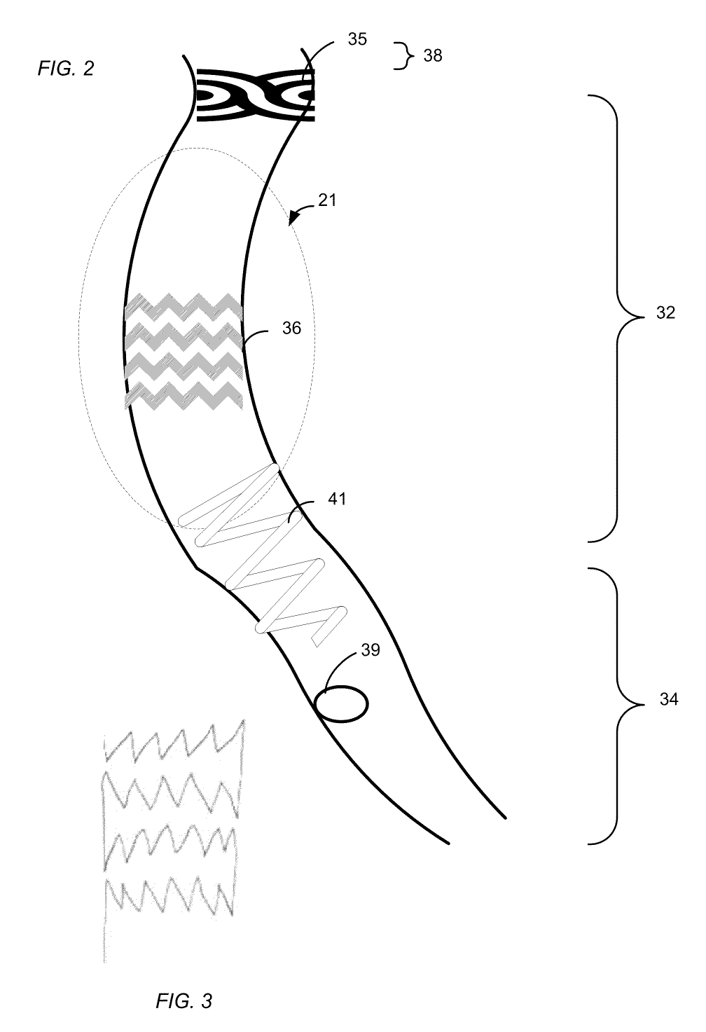

A flowchart is shown in FIG. 1 for a customized endovascular stent or graft fabrication method, and FIG. 2 shows how the parts are disposed in the region 20 of an AAA. Area 38 indicates the region below the renal arteries for which a customized stent is to be constructed. Hole 39 indicates a hole for a contralateral or a branch vessel.

Imaging modalities such as CT scanning and MRI can be used (step 12) to create three dimensional constructions of a patient's vascular anatomy. It is common for patients with AAA to have CT or MRI scans, which can be three dimensionally constructed. The three dimensional construction creates an image (step 14) of the blood flow channel 32 through the abdominal aneurysm and the bifurcation 34 of the abdominal aorta distal to the aneurysm. If the aneurysm extends below the bifurcation, the blood flow channel through this portion can also be created. For coronary vessels or cranial vessels, three dimensional imaging can be used to create a three dimensional picture of the endoluminal arterial shape. The images may be sent by the ordering physician (step 16) as a digital file over a computer network to a central fabrication center. The images received by the fabrication center can be digitally sliced into many layers (e.g., 10 layers per millimeter). The digitally sliced image or otherwise processed image (step 18) can be transferred to a stereolithography machine or other three dimensional printing devices to create a three dimensional model of the blood flow channel or endoluminal shape (step 20).

There are several different types of stereolithography or three dimensional printing. In general, a liquid or semiliquid material is hardened layer by layer. The process that initiates the hardening controls the shape of each layer. One type of stereolithography uses a liquid polymer that is hardened when irradiated with a UV laser. Each layer of the digitally sliced image is built up as the laser irradiates the surface of the polymer. A computer controls the laser and build up of each layer from the digital file.

Once a three dimensional model is created of the aneurysmal blood flow channel or endoluminal shape, a customized stent or wire scaffolding can be fabricated around the model (step 22). Because the three dimensional endoluminal model represents the shape of the endoluminal stent or stent/graft in the expanded state, methods of fabrication of the scaffolding should allow the stent to be collapsed or reduced in diameter. Preferably, the endoluminal stent or stent/graft can be mounted in or on a catheter for transarterial endovascular placement. Alternatively the customized stent and stent grafts may be placed surgically.

One method to create a custom device is to use a braiding machine that can lay down a flat or round wire braid which can conform to the unique shape of the three dimensional model. A criss-crossed braided pattern may be used. The number of crosses per inch, and the thickness of the wires, can determine the stiffness of the stent or graft. This pattern of the braid and wire size could be varied along the length of the model to provide varying stiffness and flexibility. Helical windings of flat or round wire may also be used. A representative braided system is indicated in FIG. 2 as braid 35. A representative helical system is indicated in FIG. 2 as helix 41.

Referring to FIG. 3, the wire may be formed into “V” shaped pattern 36 that may also be used to encircle the model and create a stent or stent graft. Many configurations of building a collapsible scaffolding around the three dimensional endoluminal model may be employed and are known to those skilled in the art. Representative wire materials may include stainless steel, chromium-cobalt, nickel-titanium, and polymers. Nickel titanium may be a preferable material choice as it can be heat set to better retain the shape of the model. Other methods for creating the scaffolding are to coat the model with metal through a sputter process or electro-deposition process or foil wrap. The metallized model may then be laser etched to the desired scaffolding shape. After creating the wire scaffolding around the model, the model can be dissolved, machined, etched away, or otherwise removed, leaving the scaffolding (step 24).

Post-processing may then occur (step 26). After creating the wire scaffolding, graft materials such as nylon, Teflon, or Gore-Tex may be sewn or attached to the wire scaffolding or braid. The finished product may also be drug coated. Drug coating may be performed by absorbing or adsorbing the drug onto the graft material. Alternatively, a polymer could be used to coat the metal scaffolding which may be then impregnated with drug. Holes may also be etched into regions of the scaffolding that can serve as drug reservoirs. The ends of the graph may have a ring of outward facing retention struts or hooks to help secure the device to the arterial wall.

The custom stent graft may then be packaged into a delivery catheter for endovascular placement. One design is a hollow guiding catheter into which the stent graft is placed to retain the custom device in a collapsed state. The device may then be sterilized and packaged (step 28) and return to the ordering physician for placement (step 32).

Three dimensional models of saccular aneurysms in the brain may also be created through the same process as above. The model may be dipped in a polymer to create a balloon like structure. The balloon may be folded into a catheter device for delivery. When the catheter is placed in the saccular aneurysm, the balloon device may be inflated with a polymerizable liquid polymer to exclude the aneurysm from the blood flow.

While the invention has been described with respect to certain embodiments, it should be clear to one of ordinary skill in the art, given this teaching that the invention is much broader than the embodiments shown. For example, while the system has been described in the context of the construction of an entire system, the system may be built in a modular way as well. In this case, following the modular construction, the modules or modular parts may be put together prior to or during installation. Accordingly, the description represents some, but not all, representations, and therefore the scope of this invention is to be limited only by the claims appended to this description.

What is claimed is:

1. A method for making a customized stent or stent graft, comprising the steps of:
a. obtaining a digital image of the endoluminal shape of an artery or the blood flow channel of an aneurysm;
b. processing the obtained image to create a three dimensional model of the shape or channel; and
c. fabricating a scaffold around the model such that the scaffold substantially conforms to the model.

2. The method of claim 1, wherein the digital image is three-dimensional.

3. The method of claim 1, wherein the three-dimensional model is created by stereo lithography.

4. The method of claim 1, wherein the scaffold is a wire scaffold.

5. The method of claim 1, wherein the processing includes etching.

6. The method of claim 1, further comprising sterilizing the scaffold.

7. The method of claim 1, wherein the scaffold is formed in a braided pattern.

8. The method of claim 1, wherein the scaffold is formed in a V-shaped pattern.

9. The method of claim 1, wherein the scaffold is a helix.

10. The method of claim 9, wherein the helix is formed with a wire.

11. The method of claim 10, wherein the wire is flat or round.

12. The method of claim 1, further comprising drug-coating the scaffold.

13. The method of claim 1, further comprising attaching a graft material to the scaffold.

14. The method of claim 13, wherein the graft material is selected from the group consisting of: nylon, Teflon®, and Gore-Tex®, or combinations thereof.

15. The method of claim 1, further comprising etching at least one hole into the scaffold.

16. The method of claim 15, further comprising placing a drug in the hole.

17. The method of claim 1, further comprising mounting struts or hooks to the scaffold.

18. The method of claim 1, wherein the scaffold is created in a modular fashion and the modules are connected together into a unitary component prior to or during installation in a patient.

19. The method of claim 1, further comprising installing the stent or stent graft while the scaffold is disposed on a catheter and, when the scaffold is in an installation location, expanding the scaffold such that the scaffold has a larger diameter.

20. A stent graft or stent created by the process of claim 1.

21. A computer-readable medium containing instructions for causing a computer to implement the method of claim 1.
